(19) United States
(12) Patent Application Publication
(10) Pub. No.: US 2001/0030077 A1
(43) Pub. Date: Oct. 18, 2001

(54) Stethoscope with ECG monitor

(76) Inventor: Richard L. Watson, McPherson, KS (US)

Correspondence Address: SHERMAN D PERNIA, ESQ., PC, 1110 NASA ROAD ONE, SUITE 450, HOUSTON, TX, 77058-3310, US

(21) Appl. No.: 09/758,627

(22) Filed: Jan. 10, 2001

Related U.S. Application Data
(60) Provisional application No. 60/175,355, filed on Jan. 10, 2000.

(51) Int. Cl. A61B 7/02

(52) U.S. Cl. 181/131; 381/67

(57) ABSTRACT

A electronic stethoscope head is combined with a standard stethoscope air tube headset assembly to provide a personally portable ECG stethoscope system for auscultating a living body. The electronic stethoscope head comprises a stethoscope body having a chest-bell, an air tube connector (or connectors), and a display module mounted to it. The chest-bell has a rim for contacting the body, and a base adapted to attach to the stethoscope body. An electrode assembly having at least two electrodes is disposed on the rim of the chest-bell to contact the body during auscultation and receive electrical signals from the body. The electrical signals are conducted to the display module. The display module is mounted on the stethoscope body, and receives and processes the electrical signals and display a representation of the electrical signals on a view screen, like an LCD display screen. An air passage is disposed within the stethoscope body to provide air pressure communication between the chest-bell and the air tube connector(s). The air tube connector(s) provide for attaching the electronic stethoscope head to a standard stethoscope air tube headset assembly.

### FIELD OF THE INVENTION

The present invention is in the field of apparatuses having a specific structure adapted to be placed on the living body. More specifically, the invention relates to devices detecting heart beat audio and electric signals for display in evaluating a condition of a living body.

### BACKGROUND OF THE INVENTION

The stethoscope has been an enduring instrument and symbol of the practice of western medicine for many decades. Although the classic air column stethoscope is still prevalently used for auscultation of heart and lung conditions, it has known limitations, and the field has been motivated to develop alternatives to circumvent some of the limitations. The advent in electronics of integrated micro-circuits and micro-CPUs, and advances in medical and related technologies have facilitated this development.

One of the limitations sought to be overcome is the dependance of the air column stethoscope (a simple chest-bell pickup channeling body sounds via an air column to the user's ears) on the energy contained in the sound as produced by the body for sufficient propagation of the information conveyed by the sound. The application of microphone pickups and electronic amplifiers to stethoscopes facilitated auscultation that otherwise would be limited by weak body sounds. For example, Yamada (U.S. Pat. No. 4,072,822) discloses a two-way stethoscope which permits hearing both direct and amplified sounds from the human body.

Other examples of amplified stethoscopes for auscultation of body sounds include Deno (U.S. Pat. No. 4,598,417), and Durand et al. (U.S. Pat. No. 5,602,924).

Taking a different tack to address the problem, Eisenberg et al. (U.S. Pat. No. 4,792,145) incorporate microprocessor technology in an electronic stethoscope system to provide a processed or conditioned audio representation of a detected body sound. The audio representation produced by the Eisenberg device also includes body sounds that were originally inaudible.

Although these devices may be useful for their intended purposes and may overcome some of the limitations of the classic air column stethoscope's dependance on the inherent energy contained in the original body sound, they do not address the issue that aural auscultation alone may not be sufficient to perform an adequate diagnosis, particularly of heart condition.

In view of this limitation, the field has been further motivated to develop stethoscopes that provide waveform representation, particularly of heart sounds, in addition to aural presentations. An example of such a device is disclosed by Bredesen et al. (U.S. Pat. No. 5,213,108). Bredesen describes a display module device that is electronically coupled to microphone in a stethoscope. The Bredesen device receives, digitizes and stores heart sound data, and displays waveforms of the heart sounds on an LCD screen. Such sound waveform devices may also be useful for their intended purpose, however, diagnosing a heart condition using auditory data alone, even when displayed as a waveform, still has its limitations.

In response, the field has developed electronic stethoscopes that detect and display as waveforms both audio (heart sound) and ECG (heart electrical activity), as well as presenting the heart sounds aurally. Little et al. (U.S. Pat. No. 4,362,164) describe a stethoscope having a detector head that includes a microphone and is selectably connectable to either a conventional chest-bell or an electrode chest-bell. The electrode chest-bell is adapted to pickup electric heart signals, and the microphone to pickup body sounds. The electrode and microphone signals are sent to a separate monitoring unit to display both ECG and phonocardiographic waveforms. The conventionally detected body sounds are transmitted via air column tube directly to the user's ears. However, the Little device, and others like it, require connectivity (either via interconnect cable of wireless transmission) between the stethoscope and a separate display unit, and they are not easily transported about a physician's or other care staff's person. Again, although these devices also may be useful for their intended purpose, they too have their limitations.

Therefore, it would be beneficial to have an alternative electronic stethoscope that is easily transportable about a care givers' person, and adapted for monitoring and displaying heart sounds and heart electrical activity, as well as serving the usual auscultation purposes of the conventional stethoscope.

### SUMMARY OF THE INVENTION

The present invention is personally portable ECG stethoscope system for auscultating a living body. The system is an electronic stethoscope head combined with a standard stethoscope air tube headset assembly. The electronic stethoscope head comprises a stethoscope body on which is mounted a sound signal detector (chest-bell), an electrical signal detector and a display module. The present ECG stethoscope system is easily transportable about a medical care givers' person, and adapted for monitoring and displaying heart sounds and heart electrical activity, as well as serving the usual auscultation purposes of the conventional stethoscope. The system is self-contained and does not require ancillary equipment to be connected or linked to the stethoscope to accomplish its utility.

The stethoscope body incorporates means for mounting three other components of the electronic stethoscope head: the first mounting means being a chest-bell mount adapted for receiving and mounting a chest-bell, the second mounting means being an air tube connector adapted for connecting one or more air tubes to the stethoscope body, and the third mounting means being a display module mount adapted for attaching a display module to the stethoscope body. The chest-bell serves as the sound signal detector. Typically, a chest-bell has a rim for contacting the sound detector to the living body, a base where the sound detector is mounted to the stethoscope body and through which body sounds are propagated to be sent to the air tube assembly.

The ECG stethoscope head includes an electrode assembly. The electrode assembly has at least two ECG electrodes which are incorporated into the rim of the chest-bell, and disposed to contact the dermis of living body and receive heart electrical signals during auscultation. Electrical leads connect the ECG electrodes disposed on the rim of the chest-bell with the display module and serve to conduct the heart electrical signals to the display module.

The display module is attached to or mounted on the stethoscope body by the display module mounting means. Mounting the display module to the stethoscope body can be accomplished by any of a number of means known to one of ordinary skill in the art, including using fasteners or adhesives. Alternatively, the stethoscope body is adapted to incorporate the display module, by having the stethoscope body house the display module. The display module is in electrical communication with the electrical leads of the ECG electrodes, and receives and processes the heart electrical signals from the electrodes. The display module produces display data from the electrical signals and sends the data to a view screen (e.g., an LCD screen) to provide a visual representation (waveform) of the heart electrical signals.

The air passage disposed within the stethoscope body provides air pressure communication between the interior of the chest-bell and the air tube connector mounted on the stethoscope body. The air tube connector serves as the attachment point via which the stethoscope head attaches to a standard stethoscope air tube assembly to provide air pressure communication of the body sounds to the ear pieces of the stethoscope.

The display module of the present invention comprises a housing containing a electrical signal processor and a view screen. The signal processor receives heart electrical signals, processes them and outputs screen data to the view screen. The view screen receives the screen data and presents it on the view screen. The view screen presentation includes a waveform presentation of the heart electrical signal. The electric signal processor further comprises a signal processing circuit and a power supply. The signal processing circuit is operatively connected to the electrode leads to receive heart electrical signals from the electrodes. The signal processing circuit processes the signals to produce view screen data and selectively communicates the screen data to the view screen. Which of the screen data is sent to the view screen to be presented is controlled by the signal processing circuit, and is selectable by a user via function keys on the surface of the display module proximate the view screen. A battery power supply is contained within the display module. The power supply is in operative communication with the signal processing circuit and the view screen to provide them with electrical power.

Utilizing the basic structure and elements of the present electronic stethoscope head, it is possible to include other sensor and diagnostic modalities features. For example, blood hemoglobin oxygen saturation (SAO₂) sensor technologies are presently available that can be incorporated into the rim of the chest-bell. The electrical signals from the SAO₂ sensor can be conducted to and analyzed by the signal processor and displayed on the view screen of the present invention in much the same manner as the electrical signals from the ECG electrodes.

### BRIEF DESCRIPTION OF THE DRAWINGS

FIG. 1A is a schematic representation of the personally portable ECG stethoscope system of the present invention.

FIG. 1B is a block diagram illustrating the relationship between the principle elements of the personally portable ECG stethoscope system.

FIG. 2A is a side elevation of the stethoscope head of the present invention showing the body, the chest-bell and the display module. The body includes a partial cutaway showing a pathway of the electrode wires from the bell electrodes, through the body and into the display module.

FIG. 2B is side view of the electrode assembly and signal leads removed from the drawing of FIG. 2A.

FIG. 3 is a partial cutaway through the side elevation of the stethoscope head of the present invention showing the body, the chest-bell and the display module. Also shown is the air pressure pathway from the chest-bell, through the stethoscope body and to the aural output ports that connect to the air column tubes of the stethoscope ear pieces.

FIG. 4 is a front elevation of the stethoscope head of the present invention showing first the chest-bell, with the body behind it and then the display module.

FIG. 5A is a front of a prior art display module adaptable for use in the present invention.

FIG. 5B is a block diagram of the circuit functions and general connectivity of the display module.

FIGS. 6A and 6B are side elevations of the stethoscope head showing the body serving as a housing for the display module. The body includes a partial cutaway of the bell and housing showing the air pressure pathway of the from the base of the bell, through the body housing and to the air tube connector(s).

FIG. 6C is a side elevation of the stethoscope head illustrating one of a variety of alternative chest-bells that can be practiced in the present invention.

### DETAILED DESCRIPTION OF THE INVENTION

The present invention is a personally portable ECG stethoscope system for the auscultation of a living body. The system includes an electronic stethoscope head and standard air tube assembly. Referring now to the drawings, the details of certain preferred embodiments of the present ECG stethoscope system are graphically and schematically illustrated. Like elements in the drawings are represented by like numbers, and any similar elements are represented by like numbers with a different lower case letter suffix.

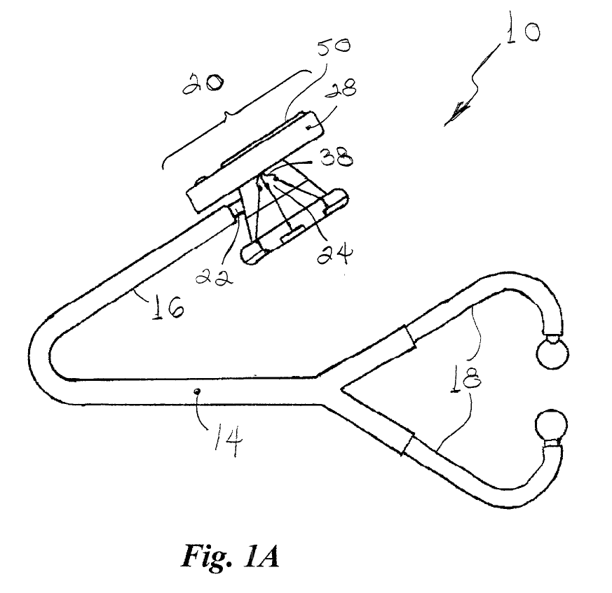

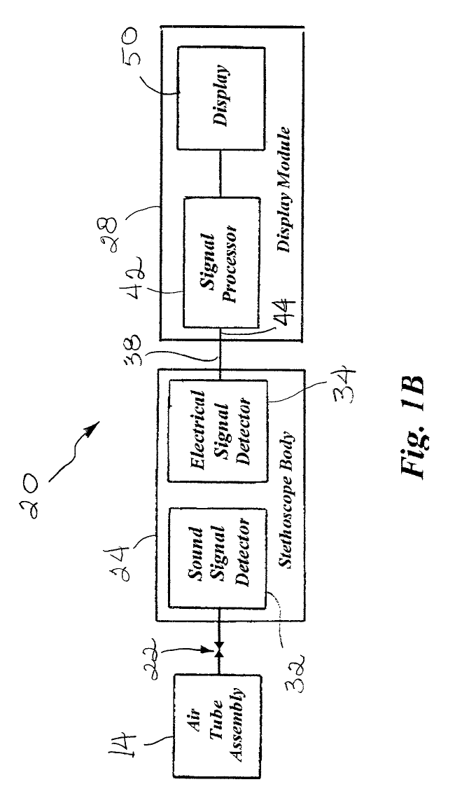

As shown in FIGS. 1A and 1B, the present personally portable ECG stethoscope system 10 is the combination of a standard aural stethoscope headset (air tube 16 and ear piece 18) assembly 14 and an electronic stethoscope head 20 connected together at an air tube connector 22. As shown in FIG. 1B, the electronic stethoscope head 20 comprises a stethoscope body 24 and a display module 28. The stethoscope body 24 includes a sound signal detector 32 and an electrical signal detector 34. Electrical signal leads 38 conduct signals from the electrical signal detector 34 to the display module 28. A signal processor 42 in the display module 28 has its input 44 in electrical communication with the signal leads 38. The signal processor 42 processes the received electrical signals into a condition suitable for input to a view screen display. The conditioned or view screen data is communicated to the view screen display 50 for visual presentation to the user.

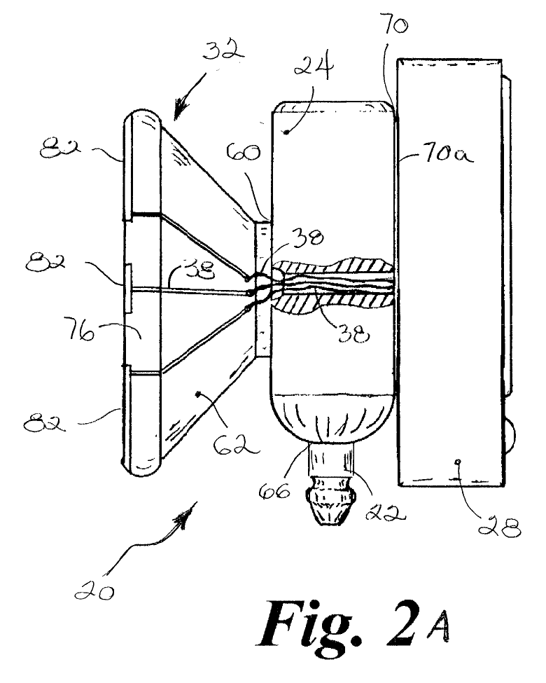

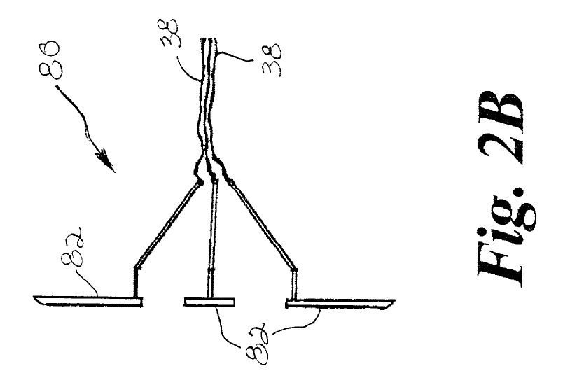

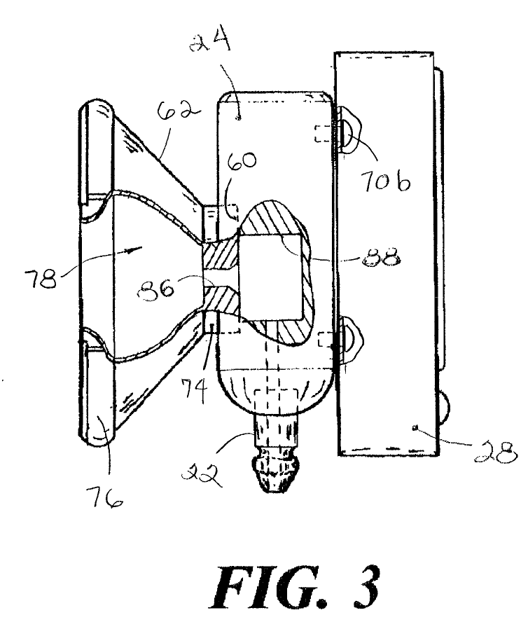

As shown in FIGS. 2A and 3, the stethoscope body 24 has first, second and third mounting means. The first mounting means 60 is adapted for receiving and mounting a sound signal detector 32 to the stethoscope body 24. The second mounting means 66 is adapted to receive an air tube connector 22 for connecting one or more air tubes 16 to the stethoscope body 24. The third mounting means 70 is adapted for attaching the display module 28 to the stethoscope body 24.

In the preferred embodiment shown in FIG. 3, the sound detector 32 (of FIG. 1B) is a chest-bell 62. The first mounting means 60 is a receptacle set into the surface of the stethoscope body 24 and receives the chest bell 62. The chest-bell 62 has a base 74, an opening rim 76, and an interior space 78 between the base 74 and the rim 76. The base 74 of the chest-bell 62 is adapted to be tightly received and held by the first mounting means 60 of the stethoscope body 24. Methods for tightly holding the base 74 in the first mount 60 are known to and adaptable by one of ordinary skill in the art for practice in the present invention. Such methods include adhesives, threaded interfaces and various screw or pin fasteners.

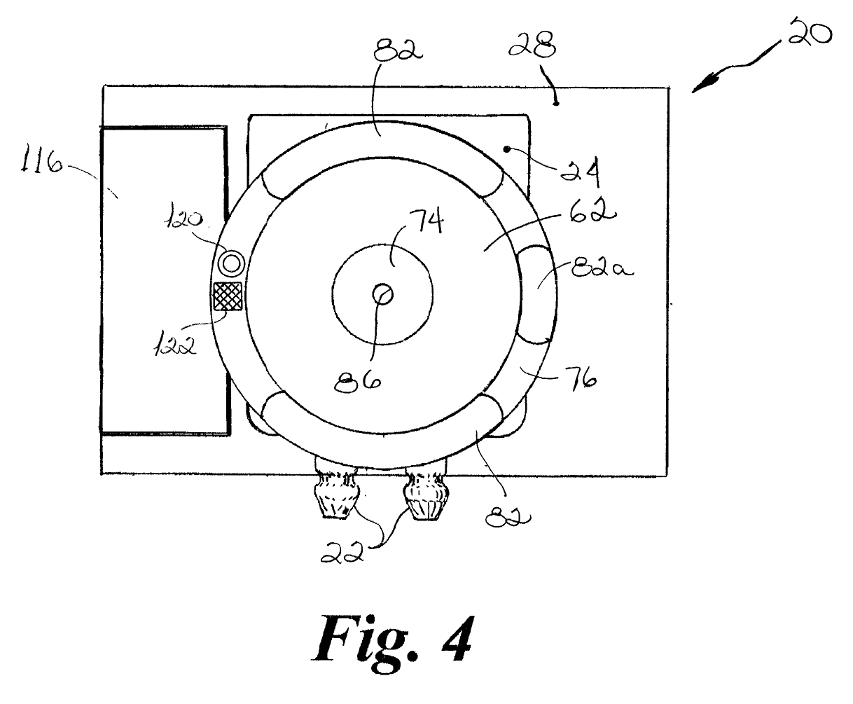

As shown in FIG. 2B, an electrode assembly 80 is associated with the chest-bell 62. The electrode assembly 80 has at least two electrodes 82. In the preferred embodiment shown in the figures, the electrode assembly 80 has three electrodes 82 which are disposed on the rim 76 of the chest-bell 62 to contact the living body during auscultation. An electrical lead 38 is connected to each electrode 82. The electrodes 82 receive heart electrical signals when the rim 76 of the chest-bell is press against the dermis over the heart region of the living body. In the preferred three electrode embodiment, one of the electrodes serves as the reference electrode 82a (see FIG. 4). The electrical leads 38 communicate the electrical signal from the electrodes 82 to the display module 28.

A display module 28 is attached to the display module mount 70 of the stethoscope body 24. The display module mounting means 70 can be accomplished by a number of means known to the ordinary skilled artisan for attaching adjoining or abutting surfaces. Examples of such means include an adhesive 70a between abutting surfaces of the stethoscope body 24 and the display module 28 as depicted in FIG. 2A, and fasteners 70b passing through one and into the other of the stethoscope body 24 and the display module as shown in FIG. 3. The display module 28 is in electrical communication with the signal electrodes 82 by way of the electrical leads 38. The electrical leads 38 are connected to the signal input 44 of the display module 28, allowing the display module 28 to receive, processes and display a waveform representation of the electrical signals on a view screen 50.

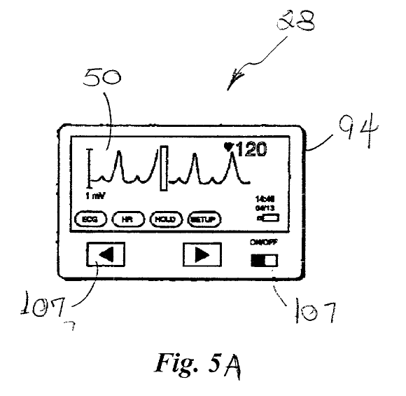

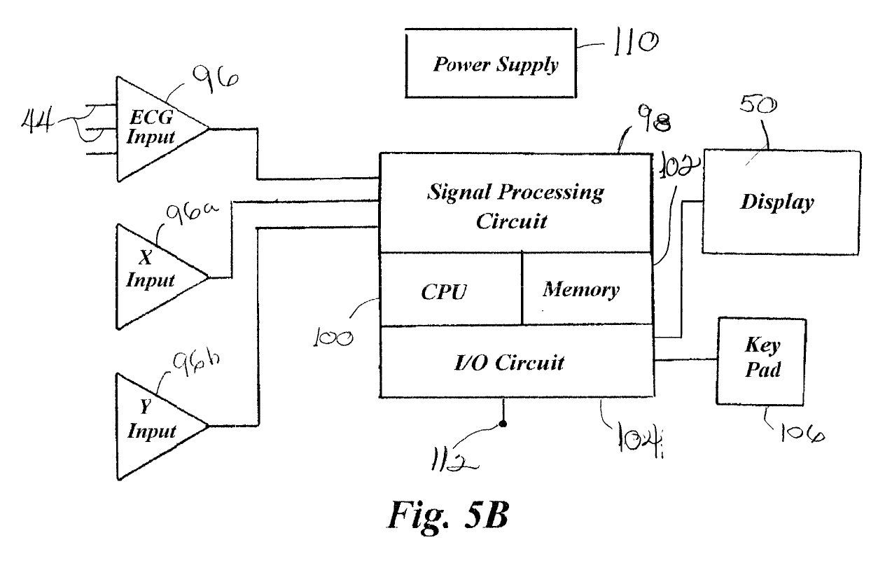

As shown in FIG. 5, the display module 28 comprises a housing 94 that contains the signal processor 42 for receiving, processing and outputting view screen data, and a view screen 50 for receiving and presenting the view screen data (see FIG. 1B). Miniature display module suitable for practice in the present electronic stethoscope head 20 are known in the art and are readily adaptable for practice in the present invention by the ordinary skilled artisan without undue experimentation. For example, Su-yeuh (U.S. Pat. No. 6,160,480) discloses a wrist watch display module for visually presenting heart rate data that it receives as a wireless input signal. A more specific example is Arcelus (U.S. Pat. No. 6,149,602), which discloses a wrist watch-like display module for visually presenting ECG data, which is also practicable in the present invention.

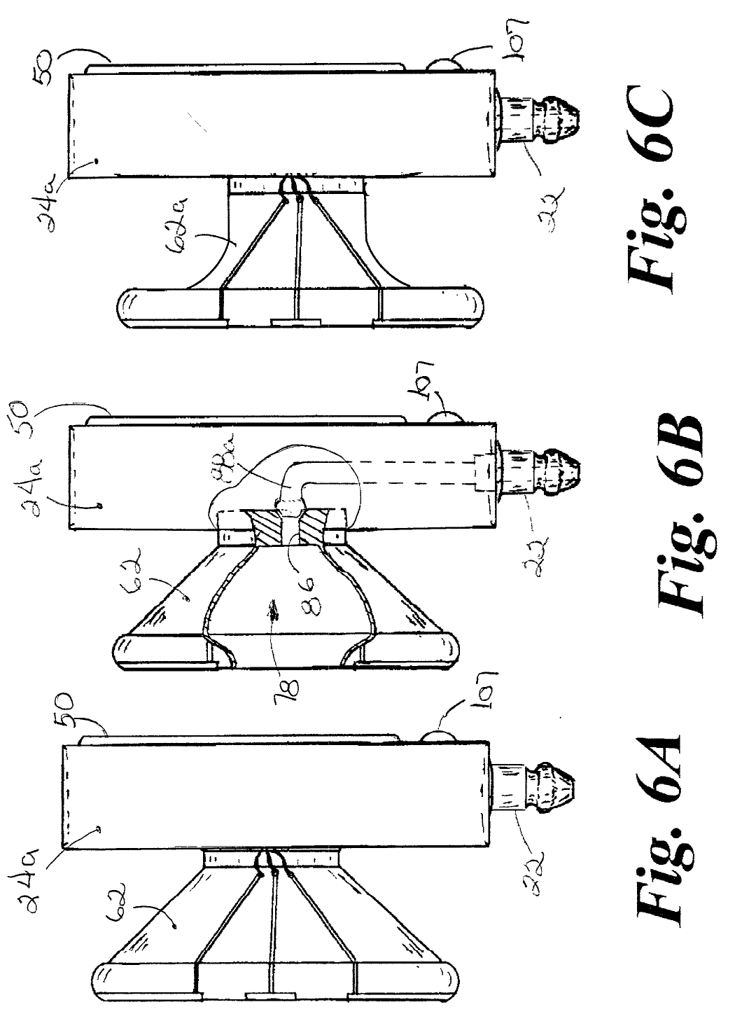

As shown in FIG. 5B, the display module 28 incorporates an ECG signal input circuit 96, including signal filter/amplifier circuitry. Optionally, other input circuits 96a, 96b for receiving electrical signals for other detectors (e.g., SAO₂ and blood sugar detectors) may be provided in the display module 28. Also included in the display module is a signal processing circuit 98, a computer processing unit (CPU) 100 with memory 102, an input/output (I/O) circuit 104, a view screen 50, and a key pad 106. A power supply 110 is in operative communication with the circuits of the display module to provide electrical power as needed. The electric signal inputs 44 to the ECG input circuit 96 are operatively connected to the electrode leads 38 of the electrode assembly 80 to receive heart electrical signals from the electrodes 82. The ECG input circuit 96 conditions the input signal as necessary and conducts it to the signal processing circuit 98 which further conditions and digitizes the heart electrical signal. The digitized heart signals are then conducted under the control of the CPU and memory circuits to the view screen display 50 for visual display, or to the I/O port 112 for export from the electronic stethoscope head 20 to a peripheral device (not shown). Peripheral devices can include printers, data storage devices, signal display equipment and other devices. While in use, the present invention does not have a physical connection to any peripheral device. Control of the signals (view screen data) conducted to the display 50 is selectable by user via function keys 107 (see FIGS. 6A-C) on the key pad 106.

In the preferred embodiment, the power supply 110 is a rechargeable power supply, and more particularly, a rechargeable battery power supply. In the preferred embodiment, the batteries of the power supply 110 are easily replaceable, and as shown in FIG. 4, a battery access hatch 116 is provided on the display module 28 to access the power supply 110 to change the battery.

In an alternative preferred embodiment shown in FIG. 6A, the stethoscope body 24 itself is adapted as the display housing 94, thus integrating the signal processor 42 and the view screen 50 with the stethoscope body 24a. FIG. 6B exemplifies how an air pressure passage 88a may be accomplished in the integrated stethoscope body 24a. Other means of integrating the signal processor 42 and view screen display 50 of the display module 28 with the stethoscope body 24 are readily accomplishable by the ordinary skilled artisan. Additionally, as shown in FIG. 6C, chest-bells 62a of various different existing configurations are also adaptable for practice in the present invention by the ordinary skilled artisan.

The basic structural features and elements of the present electronic stethoscope head 20, is adaptable to optionally include other sensor and diagnostic modalities features in the rim 76 of the chest-bell 62. Specifically, micro-sizes sensors that are operative upon contact with the dermis of a living body are especially adaptable for practice with the stethoscope head 20 of the present invention. For example, SAO₂ sensor technologies are presently available that can be incorporated into the rim of the present chest-bell 62. FIG. 4 shows the IR emitter 120 and the reflected IR detector 122 of an SAO₂ sensor mounted on the rim of the chest-bell 62. The electrical signals from an SAO₂ sensor can be conducted to and analyzed by the signal processor and displayed on the view screen of the present invention in much the same manner as the electrical signals from the ECG electrodes 82. Blood sugar sensors are another micro technology that is adaptable for practice in the present invention using the already disclosed scheme of electrical signal leads 38 communicating with the inputs 44 to the signal processor 42 of the present invention.

To provide for use of the present ECG stethoscope system 10 as a typical air column, aural stethoscope, an air passage 88 is disposed within the stethoscope body 24 to provide air pressure communication between the interior space 78 of the chest-bell 62 mounted on the stethoscope body 24 and one or more air tube connectors 22, also mounted on the stethoscope body 24. See FIG. 3 and FIG. 6B. The interior space 78 of the chest-bell 62 is in air pressure communication with the air passage 88 via an aperture 86 in the base 74 of the chest-bell 62. The air tube connector(s) 22 attaches the electronic stethoscope head 20 to a standard stethoscope air tube assembly 14, thus providing for the use of the present system 10 as a typical air column stethoscope.

While the above description contains many specifics, these should not be construed as limitations on the scope of the invention, but rather as exemplifications of one or another preferred embodiment thereof. Many other variations are possible, which would be obvious to one skilled in the art. Accordingly, the scope of the invention should be determined by the scope of the appended claims and their equivalents, and not just by the embodiments.

What is claimed is:

1. A personally portable ECG stethoscope system for auscultating a living body, the system including an electronic stethoscope head and standard air tube assembly, the electronic stethoscope head comprising:
a stethoscope body having a first a second and a third mounting means, the first mounting means being a chest-bell mount adapted for receiving and mounting a chest-bell, the second mounting means being an air tube connector adapted for connecting one or more air tubes to the stethoscope body, and the third mounting means being a display module mount adapted for attaching a display module to the stethoscope body;
a chest-bell having a base, an opening rim for contacting the living body, and an interior space between the base and the rim, with the base adapted to attach to the first mounting means of the stethoscope body;
an electrode assembly having at least two electrodes and electrical leads connected to the electrodes, the electrodes disposed on the rim of the chest-bell to contact the living body during auscultation and receive electrical signals from the living body, and the electrical leads in electrical communication with a display module to conduct the electrical signals to the display module;
a display module attached to the display module mount of the stethoscope body, the display module in electrical communication with the electrical leads of the electrodes and the display module for receiving, processing and displaying a representation of the electrical signals; and
an air passage disposed within the stethoscope body, the passage providing air pressure communication between the interior of the chest-bell mounted on the stethoscope body and the air tube connector, the air tube connector for attaching the stethoscope head to a standard stethoscope air tube assembly.

2. The electronic stethoscope head of claim 1, wherein the display module comprises a housing containing a electrical signal processor for receiving, processing and outputting view screen data, and a view screen for receiving and presenting the view screen data.

3. The display module of claim 2, wherein the stethoscope body is adapted as the housing containing the electric signal processor and the view screen.

4. The display module of claim 2, wherein the electric signal processor comprises a signal processing circuit and a power supply, the signal processing circuit operatively connected to the electrode leads to receive electrical signals from the electrodes, the signal processing circuit receiving electrical signals from the electrodes, processing the signals to produce view screen data and selectively communicating the view screen data to the view screen, and the power supply in operative communication with the signal processing circuit and the view screen to provide electrical power.

5. The electric signal processor of claim 4, wherein the power supply is a battery power supply.

6. The electric signal processor of claim 4, wherein the power supply is a rechargeable power supply.
